(19) United States
(12) Patent Application Publication
(10) Pub. No.: US 2011/0207800 A1
(43) Pub. Date: Aug. 25, 2011

(54) HIV-Dependent expression constructs and uses therefore

(76) Inventor: Yuntao Wu, Manassass, VA (US)

(21) Appl. No.: 12/320,210

(22) Filed: Jan. 21, 2009

Related U.S. Application Data
(60) Provisional application No. 61/021,630, filed on Jan. 17, 2008.

(51) Int. Cl. A61K 31/7088; C12N 5/10; C12Q 1/70; C07H 21/00; A61P 31/18

(52) U.S. Cl. 514/44 R; 435/320.1; 435/372; 435/5; 536/23.1; 536/23.7

(57) ABSTRACT

The invention provides a nucleic acid construct for targeting and killing cells infected with the human immunodeficiency virus (HIV). The construct includes an HIV Tat-dependent promoter operably linked to the coding region for a cell-killing protein, such as anthrolysin-O (anlO), which is partially or fully within an intron defined by a splice donor site and a splice acceptor site. The construct further includes a Rev Responsive Element (RRE). Therapeutic methods of treating HIV infection are also provided.

### CROSS-REFERENCE TO RELATED APPLICATIONS

This application claims the benefit of U.S. provisional patent application No. 61/021,630, filed Jan. 17, 2008, the entire contents and disclosure of which is incorporated herein by reference.

The entire disclosures and contents of each literature reference, website, patent, and patent application referred to herein are incorporated by reference.

### STATEMENT OF GOVERNMENT INTEREST

This invention was made partially with U.S. Government support from the United States National Institute of Neurological Disorders and Stroke under Contract No. R21 N2051130. The U.S. Government has certain rights in the invention.

### BACKGROUND OF THE INVENTION

1. Field of the Invention

The present invention relates to nucleic acid molecules comprising expressible sequences, including genes, whose expression is dependent on the presence of HIV proteins.

2. Description of Related Art

Acquired Immune Deficiency Syndrome (AIDS) caused by Human Immunodeficiency Virus (HIV) infection is a leading cause of illness and death in the United States and worldwide. Treatment of AIDS with available drugs is frequently ineffective due to either endogenous or acquired resistance.

The development of methods which will aid the diagnosis of HIV infection, provide a means to kill HIV infected cells, and allow the identification of new therapeutic agents for treating HIV will be of tremendous importance in AIDS treatment. Accordingly, there is an acute need in the art for such methods.

Retroviruses, such as HIV, undergo reverse transcription to form double stranded DNA, which is then integrated into the host chromatin. The integrated provirus transcribes new genomic and messenger RNAs for virion production. HIV possesses the typical three retroviral genes, gag, pol, and env, on a 9 kilobase genome. The viral genome also encodes 6 accessory or regulatory genes. The expression of this unusually high number of gene products is accomplished by use of multiple reading frames and multiple splicing sites.

The ability of Human Immunodeficiency Virus (HIV) to persist in the body has proven to be a long-standing challenge to virus eradication. Current antiretroviral therapy cannot selectively destroy infected cells; it only halts active viral replication. With therapeutic cessation or interruption, viral rebound occurs, and invariably, viral loads return to pre-treatment levels. The natural reservoirs harboring replication-competent HIV-1 include CD4 T cells and macrophages. In particular, cells from the macrophage lineage resist HIV-1-mediated killing and support sustained viral production.

The success of highly active antiretroviral therapy (HAART), marked by the drastic reduction of plasma viremia and restoration of certain immune functions [Palella F J Jr, Delaney K M, Moorman A C, Loveless M O, Fuhrer J, Satten G A, Aschman D J, Holmberg S D: Declining morbidity and mortality among patients with advanced human immunodeficiency virus infection. HIV Outpatient Study Investigators. N Engl J Med 1998, 3381:853-60; Torres R A, Barr M: Impact of combination therapy for HIV infection on inpatient census. N Engl J Med 1997, 3361:1531-2 and Battegay M I Nuesch R, Hirschel B, Kaufmann G R: Immunological recovery and antiretroviral therapy in HIV-1 infection. Lancet Infect Dis 2006, 6(5):280-7.], led initially to speculation of disease eradication in 2 to 3 years [Perelson A S, Essunger P I Cao Y, Vesanen M, Hurley A, Saksela K, Markowitz M, Ho D D: Decay characteristics of HIV-1-infected compartments during combination therapy. Nature 1997, 387{6629):188-91 and Cavert W, Notermans D W, Staskus K, Wietgrefe S W, Zupancic M I Gebhard K, Henry K, Zhang Z Q, Mills R, McDade H, Schuwirth C M, Goudsmit J, Danner S A, Haase A T: Kinetics of response in lymphoid tissues to antiretroviral therapy of HIV-1 infection. Science 1997, 276(5314):960-4.].

This original optimism was soon dampened by the realization that persistence of viral reservoirs would make it extremely difficult, if not impossible, to eradicate HIV-1 [Finzi D, Hermankova M, Pierson T, Carruth L M, Buck C, Chaisson R E, Quinn T C, Chadwick K, Margolick J, Brookmeyer R, Gallant J, Markowitz M, Ho D D, Richman D D, Siliciano R F: Identification of a reservoir for HIV-1 in patients on highly active antiretroviral therapy [see comments]. Science 1997, 278(5341):1295-300; Chun T W, Stuyver L, Mizell S B, Ehler L A, Mican J A, Baseler M, Lloyd A L, Nowak M A, Fauci A S: Presence of an inducible HIV-1 latent reservoir during highly active antiretroviral therapy. Proc Natl Acad Sci USA 1997, 94:13193-7; Wong J K, Hezareh M, Gunthard H F, Havlir D V, Ilgnacio C C, Spina C A, Richman D D: Recovery of replication-competent HIV despite prolonged suppression of plasma viremia [see comments]. Science 1997, 278:1291-5; Furtado M R, Callaway D S, Phair J P, Kunstman K J, Stanton J L, Macken C A, Perelson A S, Wolinsky S M: Persistence of HIV-1 transcription in peripheral-blood mononuclear cells in patients receiving potent antiretroviral therapy. N Engl J Med 1999,340:1614-22; Crowe S M, Sonza S: HIV-1 can be recovered from a variety of cells including peripheral blood monocytes of patients receiving highly active antiretroviral therapy: a further obstacle to eradication. J Leukoc Biol 2000, 68:345-350 and Lambotte O, Taoufik Y, de Goer M G, Wallon C, Goujard C, Delfraissy J F: Detection of infectious HIV in circulating monocytes from patients on prolonged highly active antiretroviral therapy. J Acquir Immune Defic Syndr 2000, 23(2): 114-9.].

Further identification and characterization of these reservoirs have highlighted the limitations of HAART. It has become evident that with drug cessation, viral loads return to pre-HAART levels [Davey R T Jr, Bhat N, Yoder C, Chun T W, Metcalf J A, Dewar R, Natarajan V, Lempicki R A, Adelsberger J W, Miller K D, Kovacs J A, Polis M A, Walker R E, Falloon J, Masur H, Gee D, Baseler M, Dimitrov D S, Fauci A S, Lane H C: HIV-1 and T cell dynamics after interruption of highly active antiretroviral therapy (HAART) in patients with a history of sustained viral suppression. Proc Natl Acad Sci USA 1999, 96: 151 09-14 and Hatano H, Vogel S, Yoder C, Metcalf J A, Dewar R, Davey R T Jr, Polis M A: Pre-HAART HIV burden approximates post-HAART viral levels following interruption of therapy in patients with sustained viral suppression. Aids 2000, 14:1357-63.]. With no alternative approaches in use to specifically target cells harboring the virus, it would take an estimated 60 years for some of these reservoirs to naturally decay [Finzi D, Blankson J, Siliciano J D, Margolick J B, Chadwick K, Pierson T, Smith K, Lisziewicz J, Lori F, Flexner C, Quinn T C, Chaisson R E, Rosenberg E, Walker B, Gange S, Gallant J, Siliciano R F: Latent infection of CD4+ T cells provides a mechanism for lifelong persistence of HIV-1, even in patients on effective combination therapy. Nat Med 1999, 5(5):512-517.] The primary reservoirs of HIV-1 include resting CD4 T cells and cells of the macrophage lineage. Both are the natural targets of HIV-1.

It has been shown that in vitro stimulation of resting CD4 T cells from patients receiving HAART can recover replication-competent virus. In these cells, HIV-1 exists primarily as a postintegrated latent form with no detectable viral gene expression in the absence of stimulation. Nevertheless, low-level ongoing viral replication may occur in the body even with concurrent HAART [Ramratnam B, Mittler J E, Zhang L, Boden D, Hurley A, Fang F, Macken C A, Perelson A S, Markowitz M, Ho D D: The decay of the latent reservoir of replication-competent HIV-1 is inversely correlated with the extent of residual viral replication during prolonged anti-retroviral therapy. Nat Med 2000, 6(1):82-5; Ramratnam B, Ribeiro R, He T, Chung C, Simon V, Vanderhoeven J, Hurley A, Zhang L, Perelson A S, Ho D D, Markowitz M: Intensification of antiretroviral therapy accelerates the decay of the HIV-1 latent reservoir and decreases, but does not eliminate, ongoing virus replication. J Acquir Immune Defic Syndr 2004, 35(1):33-37 and Zhu T, Muthui D, Holte S, Nickle D, Feng F, Brodie S, Hwangbo Y, Mullins J I, Corey L: Evidence for human immunodeficiency virus type 1 replication in vivo in CD14(+) monocytes and its potential role as a source of virus in patients on highly active antiretroviral therapy. J Virol 2002, 76:707-716.1.

In particular, with the cessation of therapy, rebounding plasma viruses do not entirely reflect the genetic pool of the viruses from resting CD4 T cells [Chun T W, Davey R T Jr, Ostrowski M, Shawn Justement J, Engel D, Mullins J I, Fauci A S: Relationship between pre-existing viral reservoirs and the reemergence of plasma viremia after discontinuation of highly active antiretroviral therapy. Nat Med 2000,6:757-761 and Dybul M, Daucher M, Jensen M A, Hallahan C W, Chun T W, Belson M, Hidalgo B, Nickle D C, Yoder C, Metcalf J A, Davey R T, Ehler L, Kress-Rock D, Nies-Kraske E, Liu S, Mullins J I, Fauci A S: Genetic characterization of rebounding human immunodeficiency virus type 1 in plasma during multiple interruptions of highly active antiretroviral therapy. J Virol 2003, 77:3229-32371, suggesting the existence of other reservoirs such as cells from the macrophage lineage.

In contrast to the latent reservoir of resting T cells, macrophages are metabolically active and support sustained, ongoing viral replication [Crowe S, Zhu T, Muller W A: The contribution of monocyte infection and trafficking to viral persistence, and maintenance of the viral reservoir in HIV infection. J Leukoc Biol 2003, 74:635-641.] Macrophages have minimal cytopathology in response to HIV infection and can remain viable for viral production for extended periods of time [Williams K C, Corey S, Westmoreland S V, Pauley D, Knight H, deBakker C, Alvarez X, Lackner A A: Perivascular macrophages are the primary cell type productively infected by simian immunodeficiency virus in the brains of macaques: implications for the neuropathogenesis of AIDS. J Exp Med 2001, 193:905-915 and Igarashi T, Brown C R, Endo Y, Buckler-White A, Plishka R, Bischofberger N, Hirsch V, Martin M A: Macrophage are the principal reservoir and sustain high virus loads in rhesus macaques after the depletion of CD4+ T cells by a highly pathogenic simian immunodeficiency virus/HIV type 1 chimera (SHIV): Implications for HIV-1 infections of humans. Proc Natl Acad Sci USA 2001, 981658-663].

In addition, antiretroviral drugs are poorly efficacious against chronically infected macrophages [Igarashi T, Brown C R, Endo Y, Buckler-White A, Plishka R, Bischofberger N, Hirsch V, Martin M A: Macrophage are the principal reservoir and sustain high virus loads in rhesus macaques after the depletion of CD4+ T cells by a highly pathogenic simian immunodeficiency virus/HIV type 1 chimera (SHIV): Implications for HIV-1 infections of humans. Proc Natl Acad Sci USA 2001, 98:658-663; Crowe S M, McGrath M S, Elbeik T, Kirihara J, Mills J: Comparative assessment of antiretrovirals in human monocyte-macrophages and lymphoid cell lines acutely and chronically infected with the human immunodeficiency virus. J Med Virol 1989, 29:176-180 and Perno C F, Aquaro S, Rosenwirth B, Balestra E, Peichl P, Billich A, Villani N, Calio R: In vitro activity of inhibitors of late stages of the replication of HIV in chronically infected macrophages. J Leukoc Biol 1994, 56(3):381-3861. These features suggest that macrophages are a major viral reservoir in the body [see above and Sonza S, Mutimer H P, Oelrichs R, Jardine D, Harvey K, Dunne A, Purcell D F, Birch C, Crowe S M: Monocytes harbour replication-competent, non-latent HIV-1 in patients on highly active antiretroviral therapy. Aids 2001, 15:17-221 and an attractive target for testing alternative therapies aimed at eradicating HIV reservoirs.

Clinical and experimental attempts to diminish HIV reservoirs have taken many forms. For example, infected patients have been treated with HAART plus cytokines such as IL-2 and INF-γ [Stellbrink H J, Hufert F T, Tenner-Racz K, Lauer J, Schneider C, Albrecht H, Racz P, van Lunzen J: Kinetics of productive and latent HIV infection in lymphatic tissue and peripheral blood during triple-drug combination therapy with or without additional interleukin-2. Antivir Ther 1998, 3(4):209-214; Chun T W, Engel D, Mizell S B, Hallahan C W, Fischette M, Park S, Davey R T Jr, Dybul M, Kovacs J A, Metcalf J A, Mican J M, Berrey M M, Corey L, Lane Fauci A S: Effect of interleukin-2 on the pool of latently infected, resting CD4+ T cells in HIV-1-infected patients receiving highly active antiretroviral therapy [see comments]. Nat Med 1999, 5:651-655; Lafeuillade A, Poggi C, Chadapaud S, Hittinger G, Chouraqui M, Pisapia M, Delbeke E: Pilot study of a combination of highly active antiretroviral therapy and cytokines to induce HIV-1 remission. J Acquir Immune Defic Syndr 2001, 26(1):44-55 and Dybul M, Hidalgo B, Chun W, Belson M, Migueles S A, Justement J S, Herpin B, Perry C, Hallahan C W, Davey R T, Metcalf J A, Connors M, Fauci A S: Pilot study of the effects of intermittent interleuldn-2 on human immunodeficiency virus (HIV)-specific immune responses in patients treated during recently acquired HIV infection. J Infect Dis 2002, 18561-681, or the chemical compound valproic acid [Lehrman G, Hogue I B, Palmer S, Jennings C, Spina C A, Wiegand A, Landay A L, Coombs R W, Richman D D, Mellors J W, Coffin J M, Bosch R J, Margolis D M: Depletion of latent HIV-1 infection in vivo: a proof-of-concept study. Lancet 2005, 366549-555 and Siliciano J D, Lai J, Callender M, Pitt E, Zhang H, Margolick J B, Gallant J E, Cofrancesco J Jr, Moore R D, Gange S J, Siliciano R F: Stability of the latent reservoir for HIV-1 in patients receiving valproic acid. J Infect Dis 2007, 195833-8361, in hopes of purging the latent T cell reservoir via activation-driven killing, either by the virus itself or by immune effector mechanisms.

Others have opted for more aggressive approaches to counter the HIV infected cells, such as targeting the cells with hybrid CD4-toxins that can bind to the viral envelope [Chaudhary V K, Mizukami T, Fuerst T R, FitzGerald D J, Moss B, Pastan I, Berger E A: Selective killing of HIV-infected cells by recombinant human CD4-Pseudomonas exotoxin hybrid protein. Nature 1988, 335369-3721. More recently, an HIV LTR-based lentiviral vector expressing herpes simplex virus thymidine kinase (TK) has been used to inhibit HIV replication in a latently infected T cell line [Turner L S, Tsygankov A Y, Henderson E E: StpC-based gene therapy targeting latent reservoirs of HIV-1. Antiviral Res 2006, 72:233-2411. Nevertheless, a major limitation in many of these approaches is the lack of high specificity required to target only HIV-infected cells.

### SUMMARY OF THE INVENTION

It is an object of the present invention to provide a complementary strategy to target persistently infected cells.

More specifically, it is an object of the invention to provide an HIV-1 Rev-dependent lentiviral vector carrying a cytotoxic, cytolytic or cell apoptosis inducing protein to achieve selective killing of HIV-1-infected cells.

The above and other objects are achieved by the present invention, one embodiment of which comprises an isolated nucleic acid molecule comprising:

a) a promoter, wherein the activity of the promoter is dependent on the presence of the human immunodeficiency virus (HIV) Tat protein;

b) at least one splice donor site and at least one splice acceptor site;

c) an expressible sequence which is not a wild-type HIV sequence, wherein at least part of the expressible sequence is located in an intron between the splice acceptor site and the splice donor site;

and

d) a Rev Responsive Element (RRE) from the human immunodeficiency virus,

wherein: (i) elements (a)-(d) are operably linked,
(ii) the expressible sequence comprises a therapeutic gene, or a complement thereof, and
(iii) the therapeutic gene or complement thereof encodes a cytotoxic, cytolytic or cell apoptosis inducing protein; or a protein stimulate immune response to HIV infection; or a complement thereof.

Another embodiment of the invention comprises a vector containing the above-described nucleic acid molecule.

A further embodiment of the invention comprises a host cell containing the above-described nucleic acid molecule.

Still another embodiment of the invention comprises a method of determining whether HIV is present in a sample comprising: a) contacting the above-described host cell with the sample; b) culturing the cell for an amount of time sufficient to allow HIV infection and gene expression; and c) determining whether the reporter gene is expressed by the cell; wherein expression of the expressible sequence is indicative of the presence of HIV in the sample.

A still further embodiment of the invention comprises a method of determining whether a cell is infected with HIV comprising: a) contacting the cell with the above-described vector, the vector comprising a recombinant virus; b) culturing the cell for an amount of time sufficient to allow HIV gene expression; and c) determining whether the expressible sequence is expressed by the cell; wherein expression of the expressible sequence is indicative of HIV infection of the cell.

An additional embodiment of the invention comprises a method of determining whether a subject is infected with HIV comprising: a) contacting the cells of the subject with the above-described virus vector; and b) determining whether the expressible sequence is expressed by the cells; wherein expression of the expressible sequence is indicative of HIV infection.

Another embodiment of the invention comprises a method of killing a cell infected with HIV comprising contacting the cell with the above-described virus vector.

A further embodiment of the invention comprises a method of treating a subject infected with HIV comprising administering to the subject the above described virus vector.

An additional embodiment of the invention comprises a method of determining whether a compound is capable of killing an HIV-infected cell comprising contacting the HIV-infected cell with the above-described virus vector wherein the therapeutic gene or complement thereof encodes the test compound and determining whether the encoded test compound kills the HIV-infected cell.

Another embodiment of the invention comprises a pharmaceutical composition comprising an effective amount of the above-described virus vector and a pharmaceutically acceptable carrier therefor.

### BRIEF DESCRIPTION OF THE DRAWINGS

The accompanying drawings, which are incorporated in and constitute a part of this specification, illustrate embodiments and features of the invention, and together with the written description serve to explain certain principles of the invention.

FIG. 1 shows details of the construction of the Rev-dependent lentiviral vectors. (A) Schematic representation of the Rev-dependent lentiviral constructs. Shown are pNL-GFP-RRE-SA and its derivatives. The HIV-1 5′ LTR, packaging signal (y), splice donors (D1, D4) and acceptors (A5, A7), IBES, RRE, and 3′ LTR are indicated. The Rev-dependent constructs would transcribe both spliced and unspliced transcripts as HIV-1 does. Only the unspliced or partially spliced transcripts that contain reporters or toxins are Rev-dependent for expression. α-HL is the α-hemolysin of Staphylococcus aureus. (B) Schematic representation of the HIV-1 helper construct, pCMVAR8.2, in which both the viral package signal (Ay) and the envelope gene (Env) were deleted. (C) Simultaneous expression of two genes via TRES in the Rev-dependent construct. Both the E. coli lacZ and the GFP genes were cloned into the vector, pNL-lacZ-GFP-RRE-SA, which was cotransfected with pCMVA8.2 into HEK293T cells. For comparison, pNL-GFPRRE-SA was similarly cotransfected. Shown is GFP expression measured by flow cytometry at 48 hours post cotransfection with either pNL-GFP-RRE-SA (middle panel, GFP) or pNL-lacZ-GFP-RRE-SA (right panel, LacZ-GFP). (D) Cells from pNL-LacZ-GFP-RRE-SA cotransfection were also stained for LacZ and visualized under the microscope for LacZ and GFP. One hundred cells were counted, and among them, 20 expressed both LacZ and GFP; 4 expressed only LacZ; and 76 expressed neither LacZ nor GFP.

FIG. 2 shows data indicating the specificity of the Rev-dependent lentiviral vector in HIV-1-positive T cells and macrophages. (A) Schematic representation of the three constructs used to generate the Rev-dependent GFP lentivirus. HEK293T cells were cotransfected with pNL-GFP-RRE-SA, pCMVA8.2, and the VSV-G construct. Viruses were harvested, concentrated, and used to infect a human T cell line, CEM-SS, as well as primary human macrophages. (B) Specificity of the Rev-dependent lentiviral vector in HIV-I-positive T cells. CEM-SS cells were not infected (a) or infected with NL4-3.HSA.R+E− (VSV-G) (d, 500 ng p24 per million cells), a VSV-G pseudotyped HIV-1 strain with the murine heat-stable antigen CD24 (HSA) gene inserted into the nef region that allows HIV-1-positive cells to be monitored by surface staining of HSA. At 24 hours, cells were superinfected with lentivirus vNL-GFP-RRE-SA (d, mod. 10). For comparison, cells were also singly infected with either vNL-GFP-RRE-SA (b) or NL4-3.HSA.R+E−(VSV-G) (c). At 72 hours, cells were harvested, stained with a PE-labeled rat monoclonal antibody against mouse CD24 (HSA), and then analyzed on a flow cytometer for both HSA and GFP expression. Isotype staining was not shown. (C) specificity of the Rev-dependent lentiviral vector in HIV-1-positive macrophages. Human macrophages were derived from peripheral monocytes by culturing in 10 ng/ml M-CSF for two weeks. Cells were not infected (e) or infected with HIV-1(AD8) (h, 380 ng p24 per million). At 24 hours, cells were superinfected with lentivirus vNLGFP-RRE-SA (h, m.o.i. 10). For comparison, cells were also singly infected with either vNL-GFP-RRE-SA (f) or HIV-1(AD8) (g). At 72 hours, cells were harvested and analyzed on a flow cytometer for GFP expression. (D) Fluorescent microscopy of GFP expression in macrophages infected with HIV-1 and the Rev-dependent GFP lentiviral vector. Cells in (C, h) were also examined with fluorescent microscope. The left and right panels show the bright and green fluorescent fields of the same cells. Arrows indicate an HIV-1-infected cell expressing the GFP protein.

FIG. 3 shows data indicating the extracellular and intracellular cytolytic activity of AnlO. (A) Extracellular cytolytic activity of AnlO. THP-I cells or primary human macrophages were treated with different concentrations of purified AnlO in serum-free medium for one hour at 37° C. Cytolytic activity of AnlO was assayed by measuring the relative activity of lactate dehydrogenase (LDH) released into the culture following cell lysis. (B) Inhibition of AnlO activity by human plasma. AnlO was serially diluted and incubated with human plasma (heat inactivated) for 20 minutes on ice and then added to THP-1 cells in serum-free medium for one hour at 37° C. Cytolytic activity of AnlO was assayed by measuring the relative activity of lactate dehydrogenase (LDH) released into the culture. In the controls, AnlO was directly added into THP-1 cells without being neutralized by plasma. (C) Intracellular cytolitic activity of AnlO. pNL-GFPRRE-SA (middle panels, GFP) or pNL-AnlO-GFP-RRE-SA (right panels, AnlO-GFP) was co-transfected with pCMVA8.2 into HEK293T cells. At day 2 to 4 after cotransfection, cells were analyzed by flow cytornetry for GFP expression. PI is propidium iodide. Mock transfected cells (left panels, Cell) were used as controls for the GFP and PI positive population.

FIG. 4 depicts data showing the inhibition of AnlO-mediated cytolysis of producer cells by β-cyclodextrin derivatives. (A) Structure of the membrane pore blocker 6-boc orthinine amide-β-cyclodextrin (6-BOCD). (B) Inhibition of AnlO-mediated cytolysis by 6-BOCD. HEK293T cells were cotransfected with pCMVAR8.2 and either pNL-GFP-RRE-SA (labeled as GFP) or pNL-AnlO-GFPRRE-SA (labeled as AnlO-GFP) in the absence or presence of various doses of 6-BOCD. Increases in viable GFP cells were measured by flow cytometry. (C) Lack of effect of 6-BOCD on a-hemolysin. Cells were similarly cotransfected with pCMVAR8.2 and pNL-α-HL-GFP-RRE-SA (labeled as a-HL-GFP) in the absence or presence of various doses of 6-BOCD. (D) No inhibition of 6-BOCD on viral infectivity. Lentiviral particles, vNL-GFP-RRE-SA, were generated by cotransfection of HEK293T cells with pNL-GFP-RRE-SA, pCMVAR8.2, and pCMV-VSV-G in the absence or presence of different concentrations of 6-BOCD. The resulting viral particles were used to infect an HIV-1-positive cell, J1.1 (using an equal p24 level of viruses, 150 ng p24 per million cells). The percentage of GFP positive J1.1 cells was used as an indicator for viral infectivity. (E) The Rev-dependent AnlO lentiviral vector is suicidal in HIV-1-positive cells. The HIV-1-positive cell, J 1.1, was infected with vNL-AnlO-RRE-SA (300 ng p24 per million cells). As a control, HIV-1-negative Jurkat cells were identically infected. Following infection, cells were harvested at different times and total cellular DNA was extracted and PCR amplified with primers for the anlO gene (AnlO). As a control, the DNA was also amplified with primers for the cellular β-actin pseudogene (β-actin) to ensure that the same number of cells was used.

FIG. 5 shows data indicating that there is specific targeting of HIV-1-infected macrophages by the lentiviral vector carrying anlO. (A) Schematic representation of the three constructs used to generate the Rev-dependent anlO lentivirus. HEK293T cells were cotransfected with pNL-AnlO-GFP-PRE-SA, pCMVΔ8.2, and the M-tropic envelope construct, pCAGGSSF162gp160, in the presence of 6-BOCD. Viruses were harvested, concentrated, and used to infect human macrophages. (B) Specific killing of HIV-1-positive macrophages by the Rev-dependent AnlO lentiviral vector. Human macrophages were derived from peripheral monocytes. Cells were not infected (Cell) or infected with NL4-3.HSA.R+E−(VSV-G) (m.o.i. 0.1). Following HIV infection, at 24 hours, HIV-1-infected cells were super-infected with the lentivirus vNL-AnlO-RRE-SA (approximate m.o.i. 0.5-1) or with the same lentivirus using a 10-fold higher dosage (*). HIV-1-infected cells were stained with a PE-labeled rat monoclonal antibody against mouse CD24 (HSA) and analyzed by flow cytometry at 10 days post infection with HIV-1. (C) undetectable cytolytic activity of the Rev-dependent AnlO lentiviral vector in uninfected macrophages. To determine whether vNL-AnlO-RRE-SA can nonspecifically kill uninfected macrophages, cells were similarly infected with highly concentrated virus (m.o.i. 5-10). Following infection for two weeks, cells were harvested at different times and analyzed by propidium iodide (PI) staining and flow cytometry for cytolysis. As controls, cells were also mock infected with medium, or the same dose of an empty vector virus, vNL-RRE-SA. Cells were also treated with puromycin (500 ng/ml) to induce non-specific cytolysis for the validation of PT staining and flow cytometry analysis.

FIG. 6 shows data indicating that there is selective reduction of HIV-1-infected T cells by the lentiviral vector carrying anlO. One million CEM-SS cells were first infected with the replication-competent virus NL4-3.HSA.R+ (250 ng p24 per million). Aliquots of the infected cells were then super-infected 24 hours later with two different doses of vNL-AnlO-RRE-SA (for the 1× dosage, m.o.i. 0.5-1). The extent of spreading HIV infection was measured by mouse CD24 (HSA) staining using a PE-labeled rat monoclonal antibody against HSA. Flow cytometry analyses were performed at day 5, 7, and 9 post HIV infection. Shown are the mouse CD24 staining (HSA) (y-axis) and the Forward Scatter (FSC) (x-axis) of the cells. The second dose of vNL-AnlO-RRE-SA was 100 fold (100×) higher than the first one (1×). Uninfected cells (Cell) were similarly stained and used as a control.

### DETAILED DESCRIPTION OF THE INVENTION

The present invention is based, at least in part, on the discovery of novel DNA constructs, referred to herein as “HIV dependent expression construct”, “HDEC”, or simply “expression construct” nucleic acid molecules, which comprise an expressible sequence whose expression is dependent on the presence of Rev proteins. HIV Tat regulates transcription of the expressible sequence mRNA. However, because the expressible sequence is contained, at least in part, within an intron, it is spliced out by the cellular splicing machinery unless Rev is present. Accordingly, these novel expression constructs are capable of detecting HIV infection and/or gene expression with both specificity and sensitivity. They are also useful in screening assays for compounds capable of inhibiting HIV infection and/or gene expression. They are also useful for killing HIV-infected cells through the use of cytotoxic expressible sequences.

The present invention features nucleic acid molecules comprising expressible sequences, including reporter genes and therapeutic genes, whose expression is dependent on the presence of HIV Rev proteins. Further featured are methods for detecting HIV, methods for identifying compounds that can inhibit HIV infection and/or gene expression, methods for killing HIV-infected cells, and methods of treating HIV-infected subjects.

As a particularly illustrative example of the invention, described below is a demonstration that, in the Rev-dependent lentiviral vector, expression of anthrolysin O from Bacillus anthracis (anlO) is exclusively dependent on Rev, a unique HIV-1 protein present only in infected cells. Intracellular expression and oligomerization of anlO result in membrane pore formation and cytolysis of the infected cell. Also, according to the invention, a technical hurdle in producing a Rev-dependent anlO lentivirus is overcome through the use of β-cyclodextrin derivatives to inhibit direct killing of producer cells by anlO, Using HIV-1-infected macrophages and T cells as a model, it is demonstrated below that this Rev-dependent anlO lentivirus diminishes HIV-1-positive cells.

Although the invention is illustrated below by the use of anlO as the expressible agent, it will be understood by those skilled in the art that any suitable agent may be employed; e.g., Diphtheria toxin, Botulinum toxin, Tetanus toxin, Shiga toxin, Cholera toxin, Pertussis toxin, E. coli heat-labile toxin LT, Pseudomonas Exotoxin, Tetanus toxin, Staphylococcus aureus exfoliatin B, Perffigiolysin, Listeriolysin, Alpha toxin, Streptolysin O, Leukocidin and the like. Additional agents include those described in Todar's Online Textbook of Bacteriology, Kenneth Todar, PhD, 2008 www dot textbookofbacteriology dot net slash proteintoxins dot html.

Suitable apoptosis inducing genes include, e.g., human TRAF6, Bax, BID, FasL, Gadd153/Chop and the like. Additional agents are described in www dot researchapoptosis dot coin slash apoptosis slash index dot jsp?s_cid=0001&s_src=googleppc&&gclid=CPqO2LOZiJgCFSbCDAodfDA4DQ.

Suitable proteins that stimulate an appropriate immune response include, for example, human HLA-A or HLA-B, IL-2, IL-7 and the like.

There are events in normal HIV infection that precede the accumulation of new genomic RNA. Common for host and retroviral gene expression, co-transcriptional association of the forming message with an assortment of proteins including splicing enzymes results in the removal of introns and efficient delivery of the mature message to the cytosol. The full-length HIV transcript also contains a variety of splicing donors and acceptor sites. This feature of HIV permits the encoding of various proteins in overlapping genes (within the same segment of DNA), and permits a temporal separation of gene expression. Through varied use and non-use of splicing sites, the single RNA generated from the integrated DNA can yield nearly forty different transcripts that encode a total of nine different proteins (Purcell, D. F. and Martin, M. A. (1993) J. Virol. 67:6365-78). In the infected cell, the earliest RNA generated becomes fully spliced by the cellular splicing machinery.

Fully spliced HIV transcripts encode three proteins: negative factor Nef, transactivator of transcription Tat, and the regulator of viral gene expression Rev. These three gene products are regulatory proteins that affect cellular and viral functions that lead to efficient viral replication, but more specifically, all three can alter the viral transcription output. Tat and Rev associate with regions of newly transcribing HIV RNA. Tat associates co-transcriptionally (along with numerous cellular protein factors, including an RNA polymerase II-modifying kinase) with a 5′ stem-loop structure TAR (Rana, T. M. and Jeang, K. T. (1999) Arch. Biochem. Biophys. 365: 1751 85). Tat and the associated proteins function by promoting completion of initiated transcriptional activity (processivity or anti-termination). Rev protein is responsible for the conversion from early HIV gene expression to late gene expression in the newly infected cells. Rev mediates the cytosolic delivery of singly and non-spliced message, and thus its expression coordinates the conversion of predominately Nef, Tat, and Rev (products of multiply spliced transcript) to expression of singly and unspliced HIV transcripts, such as those for the structural proteins of the virion (Pollard, V. W. and Malim, M. H. (1998) Annu. Rev. Microbiol. 52:491-532). This occurs through a physical interaction of Rev with unspliced or singly spliced transcripts and with cellular components that are responsible for message export from the nucleus.

In the pursuit of eliminating viral reservoirs, the present invention offers a solution utilizing an engineered Rev-dependent lentivirus [Wu Y, Beddall M H, Marsh J W: Rev-dependent lentiviral expression vector. Retrovirology 2007, 4:12.] to achieve high specificity. This lentiviral vector utilizes the Rev responsive element (RRE), which renders gene expression dependent on Rev, a viral early protein interacting specifically with RRE to mediate mRNA nuclear export and translation [Malim M H, Hauber J, Le S Y, Maizel J V, Cullen B R: The HIV-1 rev transactivator acts through a structured target sequence to activate nuclear export of unspliced viral mRNA. Nature 1989, 338:254-257]. Using the green fluorescent protein (GFP) as a reporter gene, this Rev-dependent vector, when assembled into a viral particle and delivered into target cells, is fully dependent on HIV with no detectable background, expression in uninfected cells [see above and Wu Y, Beddall M H, Marsh J W: Rev-dependent indicator T cell line. Current HIV Research 2007, 5(4):395-403]. However, this attractive lentivirus is unable to deliver highly effective cytotoxic or cytolytic genes into HIV-1-infected cells since these genes can directly kill the producer cells, thereby preventing lentiviral particle production in vitro.

According to the present invention, β-cyclodextrin derivatives are used to overcome this technical hurdle and successfully generate a Rev-dependent lentivirus carrying a bacterial cytolytic gene, Anthrolysin O (anlO), and used to target HIV-1-infected macrophages. anlO is a thiol-activated hemolysin from the bacterium Bacillus anthracis. The thiol-activated hemolysins are a family of cytolysins expressed by 15 diverse bacterial species. Features common to these hemolysins include inhibition by free cholesterol and the presence of a unique cysteine residue that renders the hemolysins susceptible to reverse inactivation by oxidation. The mechanism of hemolysin action is thought to involve an oligomerization of 20 to 80 monomers into ring and arch-like structures that aggregate within the cell membrane and form large pores [Gilbert R J, Rossjohn J, Parker M W, Tweten R K, Morgan P J, Mitchell T J, Errington N, Rowe A J, Andrew P W, Byron O: Self-interaction of pneumolysin, the pore-forming protein toxin of Streptococcus pneumoniae. J Mol Biol 1998, 284:1223-1237 and Shatursky O, Heuck A P, Shepard L A, Rossjohn J, Parker M W, Johnson A E, Tweten R K: The mechanism of membrane insertion for a cholesterol-dependent cytolysin: a novel paradigm for pore-forming toxins. Cell 1999, 99:293-2991. The most compelling evidence for a direct role of thiol-activated lysins in cell killing came from studies on Listeriolysin O (LLO) in Listeria monocytogenes infection. It has been shown that a PEST-like motif at the N-terminus of LLO is responsible for its unique ability to lyse phagosomal but not cytoplasmic membrane [Decatur A L, Portnoy D A: A PEST-like sequence in listeriolysin O essential for Listeria monocytogenes pathogenicity.[see comment]. Science 2000, 290:992-9951. Upon their release from the phagosome, PEST-containing lysins are rapidly degraded by the cellular protein degradation pathway, preventing the lysins from attacking the host cell membrane and allowing the microbe to establish a productive intracellular infection. In contrast, mutants that lack the PEST-like sequence enter the host cytosol but subsequently permeabilize and kill the host cell. The LLO analog, anlO, expressed by B. anthracis is highly homologous to LLO (37% identity). The ability to escape the phagosome of macrophages is also a characteristic feature of B. anthracis. However, in contrast to the non-cytolytic nature of L. monocytogenes infection, B. anthracis infection results in the death of infected macrophages. Consistently, the anlO sequence contains no PEST homology, and anlO kills macrophages likely by direct lysis of the cell membrane.

The unique features of LLO have been used for the delivery of gelonin toxin into tumor cells for therapeutic purposes [Provoda C J, Stier E M, Lee K D: Tumor cell killing enabled by listeriolysin O-liposome-mediated delivery of the protein toxin gelonin. J Biol Chem 2003, 278:35102-35108]. In in vitro experiments, co-encapsulated LLO enabled the release of liposomal gelonin into cell cytoplasm, resulting in rapid cell killing by gelonin. Conceivably, in this Rev-dependent lentiviral system, anlO would be superior to LLO, because cytosolic anlO can cause cell death even in the absence of gelonin.

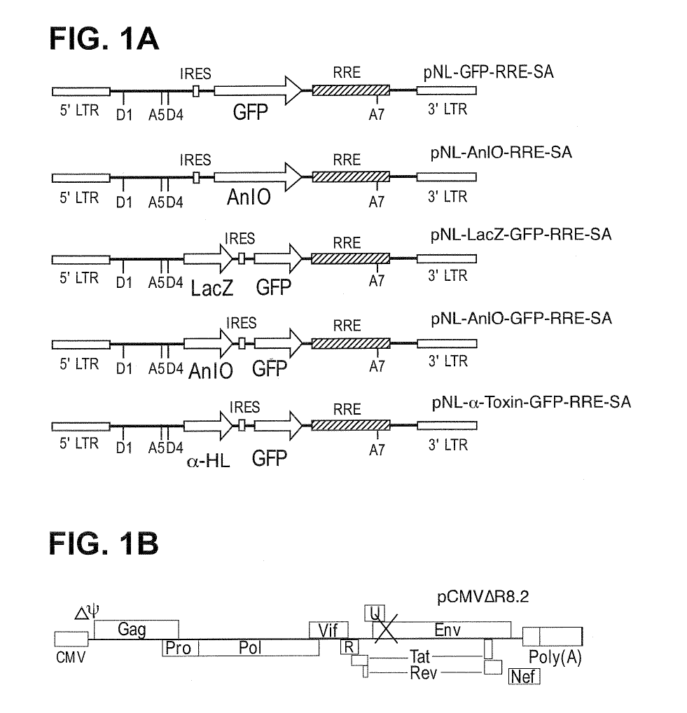

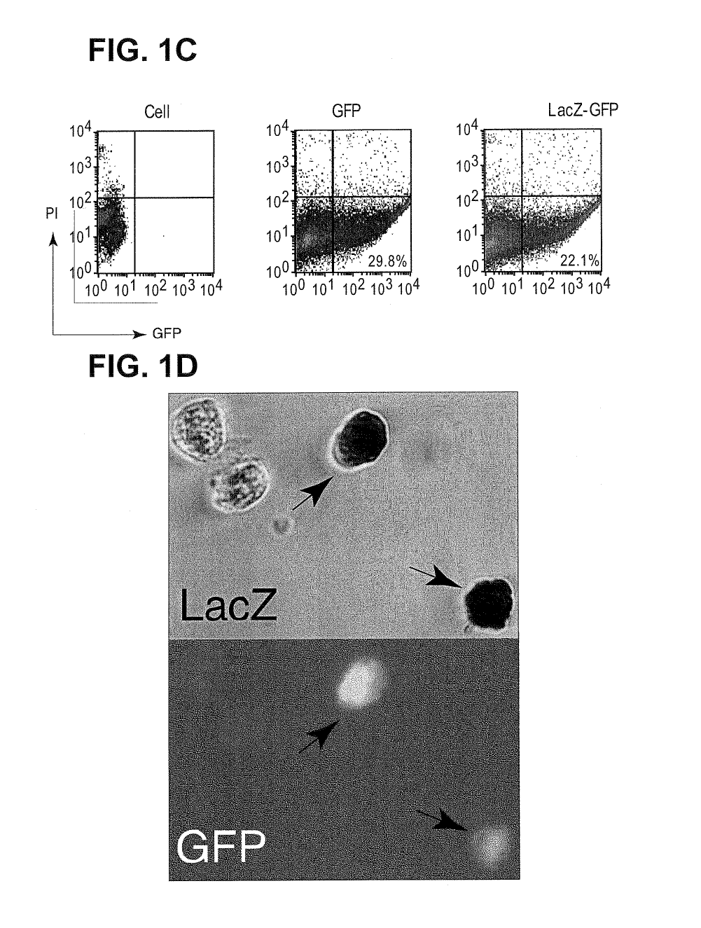

The Rev-dependent lentiviral vector is structurally based upon the HIV-1 genome and has been described previously (FIG. 1A). As shown in FIG. 1A, the GFP or the anlO gene is placed under the control of Rev by introducing multiple splicing sites and an RRE. This arrangement would regulate these genes as late genes and render their expression highly specific to Rev. The lentiviral vector also contains an internal ribosome entry site (IRES) that allows the expression of two genes simultaneously. To demonstrate the operation of the TRES, both the E. coli lacZ and the GFP gene were inserted into a single vector. This construct, pNL-LacZ-GFP-RRE-SA (FIG. 1A), was then cotransfected with an HIV-1 helper plasmid, pCMVΔ8.2 (FIG. 1B) [Naldini L, Blomer U, Gallay P, Ory D, Mulligan R, Gage F H, Verma T M, Trono D: In vivo gene delivery and stable transduction of nondividing cells by a lentiviral vector. Science 1996, 272:263-267], which provides both Tat and Rev to mediate the expression of LacZ and GFP in the same cell. Indeed, in the cotransfected HEK293T cells, co-expression of LacZ and GFP was detected with the help of pCMVΔ8.2 {FIGS. 1C and 1D). The IRES feature was also implemented in this study to clone anlO in front of GFP (pNL-anlO-GFP-RRE-SA). This would permit monitoring of anlO-mediated lysis of HIV-positive cells by directly measuring the reduction of GFP expression; because only 30 molecules of anlO are required to trigger cytolysis [Shannon J G, Ross C L, Koehler T M, Rest R F: Characterization of anthrolysin O, the Bacillus anthracis cholesterol-dependent cytolysin. Infection & Immunity 2003, 71: 3183-3189], GFP would not be able to accumulate in cells that also express anlO.

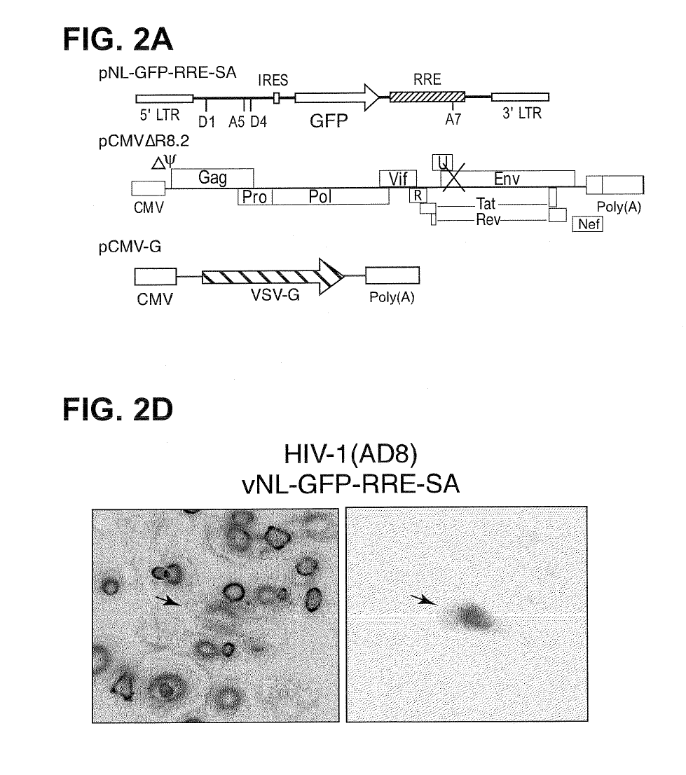

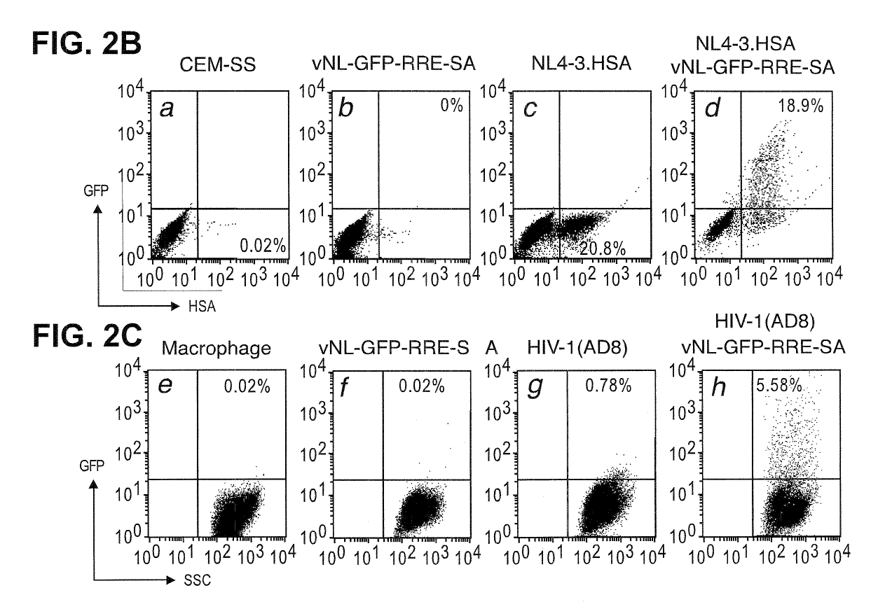

Previously, using GFP as a reporter, it was demonstrated that the Rev-dependent lentivirus can mark 80-90% of HIV-1-infected cells. To further test the specificity of the Rev-dependent vector to express genes both in primary human macrophages and in T cells, a Rev-dependent GFP lentivirus, vNL-GFP-RRE-SA wad produced and tested. Viral particles were generated by cotransfection of HEK293T cells with pNL-GFP-RRE-SA, pCMVΔ8.2, and a construct expressing the VSV-G envelope (FIG. 2A). Concentrated particles were then used to infect a human T cell line, CEM-SS, or human monocyte-derived macrophages, either directly or following infection with HIV-1. For HIV-1 infection, CEM-SS were infected with NL4-3. HSA.R+E− (Vpr⁺, Env⁻), a VSV-G pseudotyped HIV strain with the murine heat stable antigen CD24 (HSA) gene inserted into the nef region to facilitate surface HSA staining of HIV-1-positive cells [He J, Choe S, Walker R, Di Marzio P, Morgan D O, Landau N R: Human immunodeficiency virus type 1 viral protein R (Vpr) arrests cells in the G2 phase of the cell cycle by inhibiting p34cdc2 activity. J Virol 1995, 69(11):6705-6711]. Macrophages were similarly infected with an M-tropic virus, HIV-1 (AD8) [Englund G, Theodore T S, Freed B O, Engleman A, Martin M A: Integration is required for productive infection of monocyte-derived macrophages by human immunodeficiency virus type 1. J Virol 1995,69(5):3216-3219]. As shown in FIG. 2B to 2D, while vNL-GFP-RRE-SA effectively targeted HIV-1-infected cells, this lentiviral vector did not generate any GFP-positive cell without HIV-1 coinfection even with a vector multiplicity of infection as high as 10.

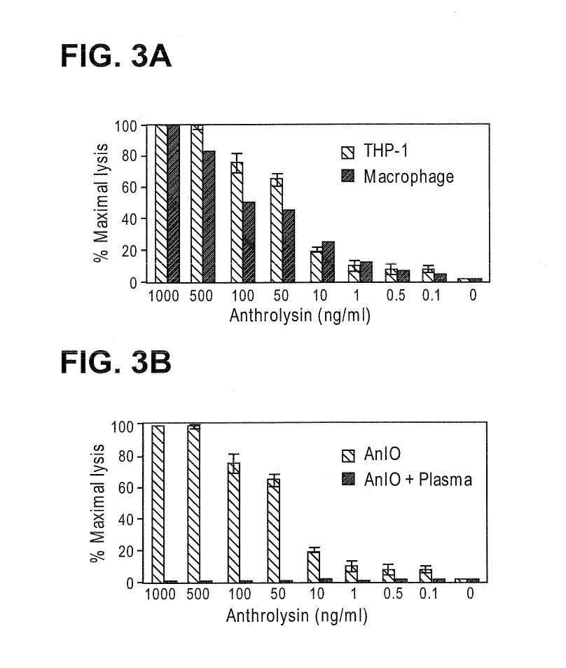

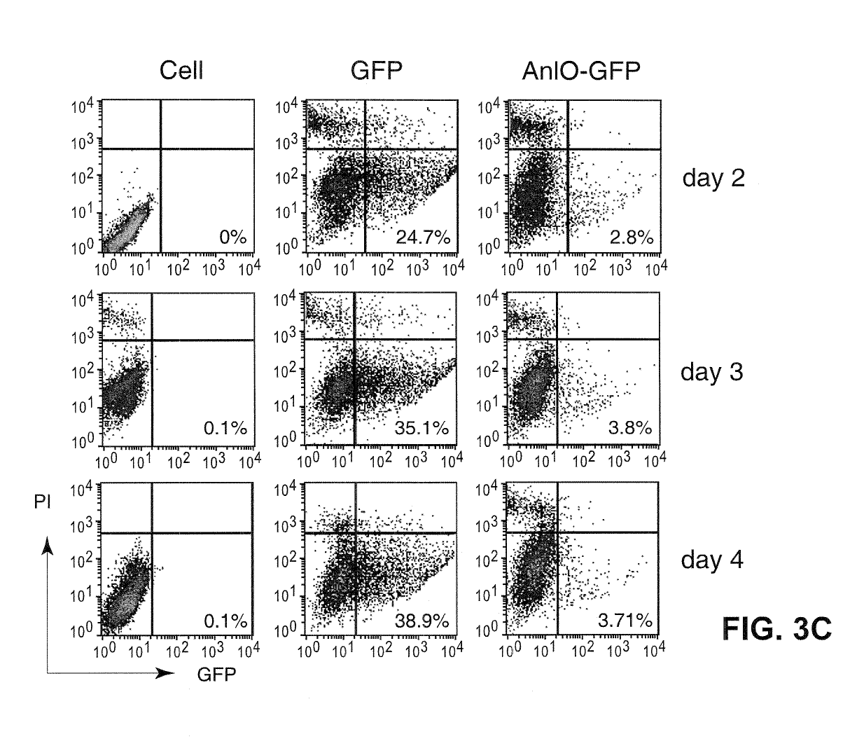

To further test cytolysis by intracellular delivery of AnlO, via the Rev-dependent lentiviral vector, cells were cotransfected with the HIV-1 helper construct, pCMVΔR8.2, and either pNL-AnlO-GFP-RRE-SA or a control plasmid, pNL-GFP-RRESA (FIG. 3C). The degree of cell lysis from anlO expression was measured by comparing GFP expression in the two parallel cotransfection procedures. As mentioned above, the reduction in GFP positive population was used as an indicator for AnlO-mediated cytolysis. As shown in FIG. 3C, at day 2, cells cotransfected with pNL-AnlO-GFP-RRE-SA generated a much lower percentage (AnlO-GFP, 2.8%) of GFP positive cells than the control cells that were cotransfected with pNL-GFP-RRE-SA (GFP, 24.7%). Additionally, the GFP intensity in cells cotransfected with AnlO-GFP was also lower (mean GFP intensity: 352.99 versus 1397.02). At days 3 and 4, little increase was observed in the number of GFP-positive cells cotransfected with AnlO-GFP (from 2.8% to 3.8%). In contrast, a significant increase of GFP positive cells was detected in the control cotransfection (from 24.7% to 38.9%) (FIG. 3C). Similar results were obtained from three independent co-transfection experiments. The diminished GFP expression in pNL-AnlO-GRP-RRE-SA cotransfection did not result from differences in cotransfection efficiency or from reduced gene expression mediated by IRES, as demonstrated in three ways. Firstly, measurements of the amount of plasmid DNA extracted from cells immediately following cotransfection revealed no significant difference from that of the control cotransfection. Secondly, the addition of β-cyclodextrin derivatives, which block cell membrane pores and inhibit AnlO mediated cytolysis [Karginov V A, Nestorovich E M, Moayeri M, Leppla S H, Bezrukov S M: Blocking anthrax lethal toxin at the protective antigen channel by using structure-inspired drug design. Proc Natl Acad Sci USA 2005, 102:15075-15080; Karginov V A, Yohannes A, Robinson T M, Fahmi N E, Alibek K, Hecht S M: Beta-cyclodextrin derivatives that inhibit anthrax lethal toxin. Bioorg Med Chem 2006, 14:33-40], led to a significant increase in the GFP positive cells in pNL-AnlO-GFP-RRE-SA cotransfection (see below), whereas the same compound had little effect on cells cotransfected with the control plasmid (pNL-GFP-RRE-SA). Thirdly, with the cloning of multiple genes and toxins into the Rev-dependent vector, it was consistently observed that diminished expression of GFP from IRES always closely correlated with the cytotoxicity of co-expressed genes. For example, when lacZ was co-expressed with GFP via IRES, an approximately equal number of GFP-positive cells was observed regardless of the presence of the lacZ gene. In contrast, when a highly cytotoxic gene, diphtheria toxin (DT) (one molecule would kill a cell) [Yamaizumi M, Mekada E l Uchida T I Okada Y: One molecule of diphtheria toxin fragment A introduced into a cell can kill the cell. Cell 1978, 15:245-2501, was placed in front of GFP, not a single GFP-positive cell was detected. Additionally, when the same DT constructs were cotransfected into a diphtheria toxin-resistant cell line, an equal number of GPF positive cells were observed regardless of the presence of the DT gene. Based on these observations, it is concluded that similar to the DT cotransfection, the diminished expression of GFP in pNL-AnlO-GFP-RRE-SA cotransfection correlates with AnlO-mediated cytolysis.

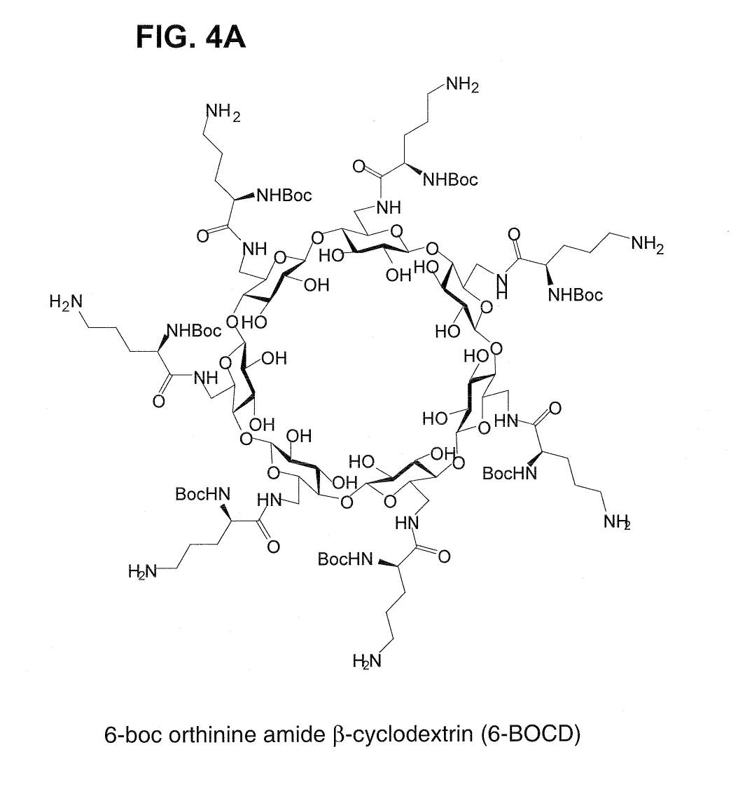

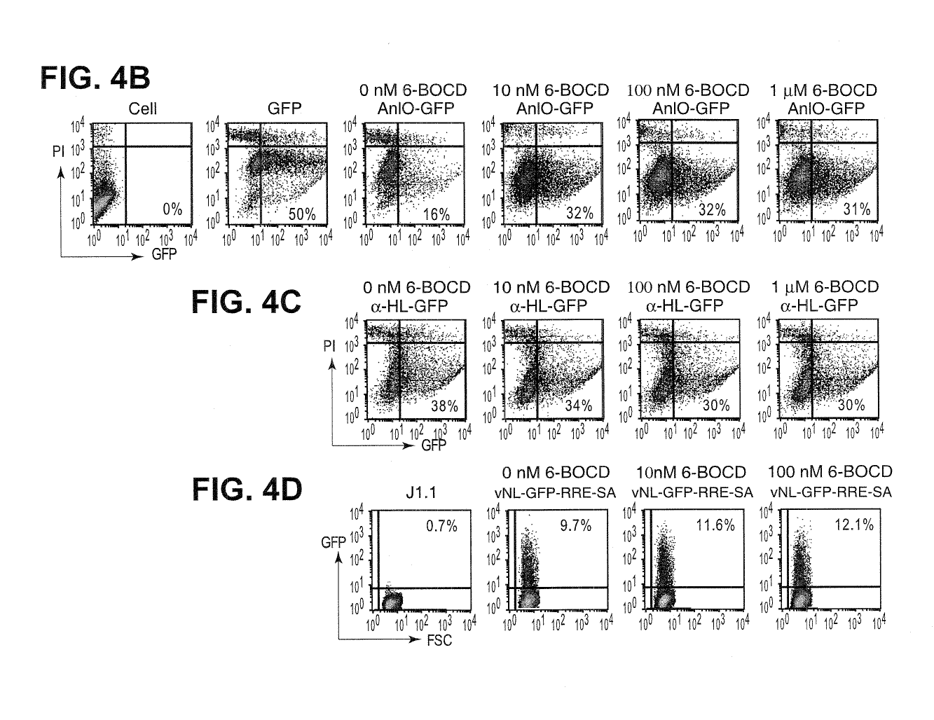

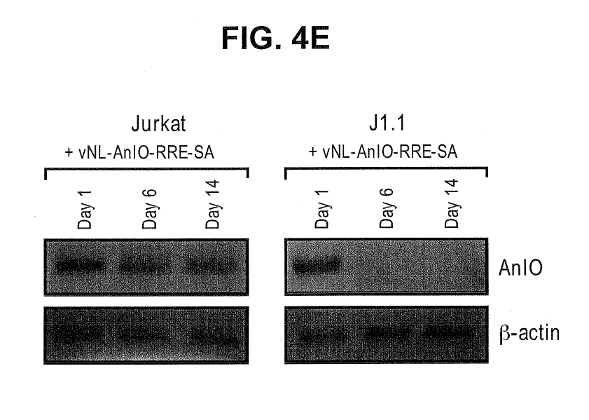

Production of lentiviral particles carrying the anlO gene—The demonstration of the intracellular cytolytic activity of AnlO suggested a possible application of AnlO in killing HIV-1-positive cells. However, its cytolytic activity also presented an immediate problem in lentiviral production. Cotransfection with pCMVΔR8.2 is required to produce lentiviral particles, but the expression of Rev in turn allows the expression of anlO from the lentiviral construct. This would result in the lysis of HEK293T producer cells, diminishing viral production. To solve this problem, advantage was taken of knowledge that β-cyclodextrin derivatives can partially block ion conductance through pores formed by hemolysins [Karginov V A, Nestorovich E M, Moayeri M, Leppla S H, Bezrukov S M: Blocking anthrax lethal toxin at the protective antigen channel by using structureinspired drug design. Proc Natl Acad Sci USA 2005, 102:15075-15080; Karginov V A, Yohannes A, Robinson T M, Fahmi N E, Alibek K, Hecht S M: Betacyclodextrin derivatives that in hibit anthrax lethal toxin. Bioorg Med Chem 2006, 14:33-40]. Thus, β-cyclodextrin derivatives were tested for their ability to block the plasma membrane pores induced by AnlO. Three β-cyclodextrin derivatives were tested: 6-thioethylamino-β-cyclodextrin hydrochloride, 6-thiohexylamino-β-cyclodextrin hydrochloride, and 6-bac orthinine amide-β-cyclodextrin (6-BOCD) (FIG. 4A). Cells were cotransfected with pCMVΔR8.2 and pNL-anlO-GFP-RRESA in the presence of various concentrations of β-cyclodextrin derivatives. It was found that 6-BOCD had the best effects on inhibition of cytolysis by anlO, resulting in a doubling of the GFP-positive cell population (FIG. 4B).

Another hemolysin, the α-hemolysin (α-HL) of Staphylococcus aureus, was also tested in the Rev-dependent lentiviral vector (FIG. 1A). Interestingly, although both anlO and α-HL can form transmembrane pores, α-HL was less effective in mediating intracellular killing in comparison with anlO (FIG. 4C). The reason is not clear, but could result from the lack of an intracellular receptor for the oligomerization of α-HL which is normally delivered extracellularly [Bhakdi S, Tranum-Jensen. J: Alpha-toxin of Staphylococcus aureus. Microbial Rev 1991, 55(4):733-751; Masser E M, Rest R F: The Bacillus anthracia cholesteroldependent cytolysin, Anthrolysin O, kills human neutrophils, monocytes and macrophages. BMC Microbiol 2006, 6:56]. Consistent with a lack of cell lysis by intracellular α-hemolysin, similar 6-BOCD treatment of cells cotransfected with the α-hemolysin construct (pNL-α-HL-GRP-RRE-SA) did not increase the number of GFP positive cells (FIG. 4C).

The impact of 6-BOCD on the infectivity of the resulting lentivirus was also tested (FIG. 4D). Infectious vNL-GFP-RRE-SA was generated by cotransfection of pNL-GFP-RRE-SA, pCMVΔR8.2, and a construct expressing the VSV-G envelope protein in the presence or absence of different concentrations of 6-BOCD. The resulting lentiviruses were used to infect the HIV-1-positive J1.1 cell line [Perez V L, Rowe T, Justement J S, Butera S T, June C H, Folks T M: An HIV-1-infected T cell clone defective in IL-2 production and Ca2+ mobilization after CD3 stimulation. J lmmun 1991, 147(9):3145-3148], using an equal level of p24. GFP expression in 71.1 cells was used to measure viral infectivity. The vNL-GFP-RRE-SA virus produced in the absence of 6-BOCD generated 9.69% GFP-positive cells, whereas the same virus generated in the presence of 6-BOCD at various doses (from 10 to 100 nM) showed no difference in infectivity (FIG. 4D). Therefore, a combined method of treating the producer cells with 6-BOCD and concentrating the virus through anion exchange columns and size-exclusion columns was used to produce high titer virus despite the cytotoxicity of anlO. It was possible to produce liters of the anlO lentivirus (vNL-AnlO-RRE-SA, VSV-G pseudotyped) and concentrate them to several milliliters for the infection of HIV-1-positive cells.

To further confirm that vNL-anlO-RRE-SA was indeed a suicidal vector in HIV-1-positive cells, this lentivirus was used to infect the HIV-1-positive J1.1 cells, and the persistence of this vector in J1.1 cells was then followed. As a control, vNL-anlO-RRE-SA was also used to infect the HIV-1-negative parental Jurkat cells. Following infection, cells were harvested at different times, and then total cellular DNA was extracted, and PCR-amplified for the detection of the anlO lentiviral vector in these cells. As shown in FIG. 4E, while vNL-anlO-RRE-SA persisted for as long as two weeks in the HIV-1-negative Jurkat cells, it diminished within 6 days in the infection of the HIV-1-positive J1.1 cells. These data suggest that cytolysis mediated by anlO in HIV-1-positive cells likely led to the self-destruction of the anlO vector. Additionally, the results that vNL-anlO-RRE-SA was maintained in HIV-1-negative cells for weeks (FIG. 4E) or even months further demonstrated that possible HIV-1-independent expression of anlO was minimal in uninfected cells.

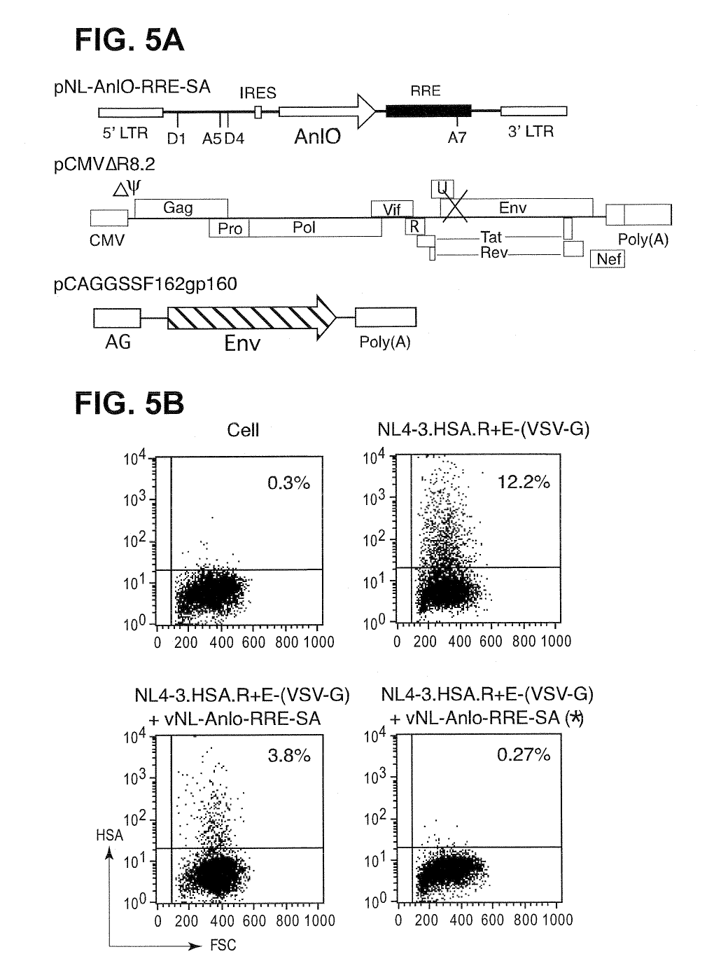

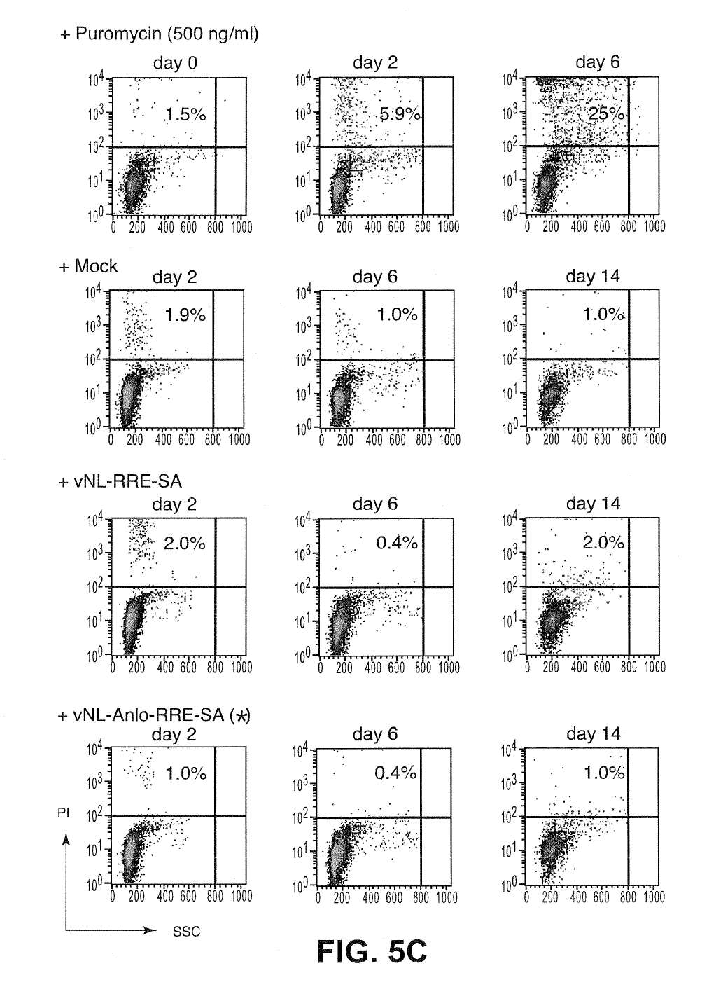

Specific killing of HIV-1-infected macrophages by the Rev-dependent lentiviral vector carrying anlO—To determine whether the Rev-dependent lentivirus carrying anlO is effective in targeting HIV-1-infected primary human macrophages, concentrated vNL-anlO-RRE-SA was produced in the presence of 10 nM 6-BOCD by cotransfection of pNL-anlO-RRE-SA, pCMVΔR8.2, and an M-tropic HIV envelope construct, pCAGGSSF162gp160 [Cheng-Mayer C, Liu R, Landau N R, Stamatatos L: Macrophage tropism of human immunodeficiency virus type 1 and utilization of the CC-CKR5 coreceptor. J Virol 1997, 71 (2):1657-1661] (FIG. 5A). To demonstrate specific killing, macrophages were first infected with NL4-3.HSA.R+E− (Vpr+, Env⁻), a strain with the murine heat-stable antigen CD24 (HSA) gene inserted into the nef region to facilitate the identification of HIV-1-positive cells by surface CD24 staining. NL4-3.HSA.R+E− was also pseudotyped with the VSV-G envelope to limit viral replication to a single round so that analysis could be performed by limiting cytolysis. The HIV-1-infected cells were further superinfected at 24 hours with the lentivirus vNL-anlO-RRE-SA. As shown in FIG. 5B, HIV-1-infected macrophages without vNL-anlO-RRE-SA superinfection generated 12.2% HIV-1-positive cells, while cells superinfected with vNL-anlO-RRE-SA showed a reduction in HIV-1-positive cells to 3.8%. Additionally, HIV-1-infected macrophages treated with a concentrated dose of vNL-anlO-RRE-SA generated only 0.27% of HIV-1-positive cells, demonstrating that the anlO lentivirus has the capacity to diminish the HIV-1-positive cell population. The direct killing of HIV-1-positive macrophages was also consistent with a decrease in the p24 level in the culture supernatant.

The selective reduction of HIV-1-positive macrophages did not result from possible non-specific killing of macrophages by the anlO lentiviral vector. When healthy macrophages were identically treated with the concentrated vNL-anlO-RRE-SA, or with a control empty vector virus, vNL-RRE-SA, differences in cytolysis were not observed during a window of two weeks, based on propidium iodide (PI) staining and flow cytometry analysis (FIG. 5C). In contrast, in a control, when puromycin (500 ng/ml) was added into the cell culture to induce nonspecific killing, cytolysis was quickly detected within 2 days, and it reached 25% of the cells at day 6 (FIG. 5C).

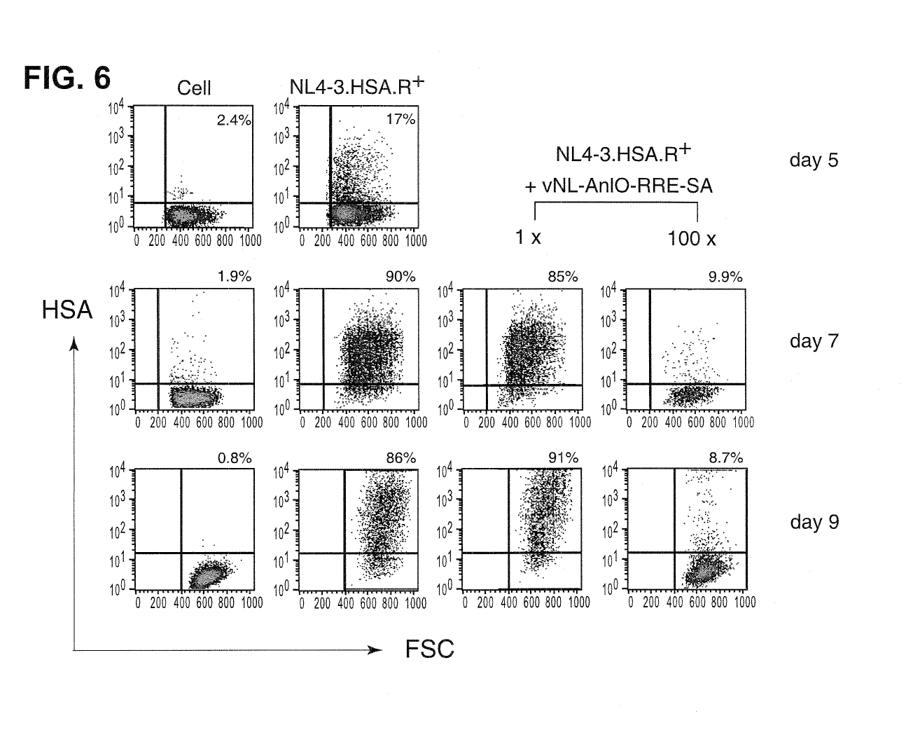

The study was further extended to test whether the Rev-dependent anlO lentivirus is capable of killing infected T cells and inhibiting viral spreading. A human CD4 T cell line, CEM-SS, was first infected with a replication-competent virus, NL4-3.HSA.R+ (Vpr⁺, Env⁻). Following infection for 24 hours, cells were superinfected with two different doses of the Rev-dependent AnlO lentivirus, vNL-anlO-RRE-SA(VSV-G). Cells were continuously cultured for more than a week, and HIV-1 spread was monitored by surface staining of mouse CD24 expression. As shown in FIG. 6, HIV-1 replication resulted in an infection spreading to 90% of T cells within one week. Super-infection with the low dose of vNL-anlO-RRE-SA(VSV-G) was not capable of competing with the virus and inhibiting its replication. However, at the high dosage (100-fold), vNL-anlO-RRE-SA(VSV-G) effectively limited the HIV spread to below 10% of T cells (FIG. 6). These results demonstrate that the Rev-dependent anlO lentivirus is capable of inhibiting HIV-1 spread by killing of infected cells. The data also demonstrate that AnlO is an effective toxin both in macrophages and in T cells.

The present invention establishes a system to specifically target HIV-1-infected macrophages and T cells by utilizing a Rev-dependent lentiviral vector carrying anlO. Lentiviruses are unique in their capacity to infect terminally differentiated cells such as macrophages. The Rev-dependency of this vector further limits anlO expression to HIV-1-positive cells. The choice of anlO as the primary therapeutic gene is based on the demonstration that anlO exhibits cytolytic activity in macrophages, and that this cytolytic activity can be inhibited by the presence of small quantities of cholesterol such as those present in human plasma. These properties make anlO an attractive candidate for the safe use of suicidal viral vectors with minimal secondary effects on non-target cells. It is demonstrated herein that selective killing of HIV-1-infected cells is achieved with this lentiviral vector while retaining the healthy cell population.

Host cells according to the invention are exemplified by human T cells carrying the lentiviral vector NL-GFP-RRE-SA (Wu, et al. Rev-dependent indicator T cell line, 2007, Current HIV Research, 5:394-402). When HIV infects this cell, GPF (green fluorescent protein) is expressed so that the cell can indicate the presence of HIV-1.

It will be understood by those skilled in the art that the present invention also includes the simultaneous targeting of multiple viral reservoirs, since macrophages are not the only cells harboring HIV. CD4 T cells are the other major targets of HIV-1. Although productive HIV-1 replication directly kills CD4 T cells, it has been well-documented that some infected T cells can survive virus-mediated killing and revert to a resting memory T cell phenotype [Lassen K, Han Y, Zhou Y, Siliciano J, Siliciano R F: The multifactorial nature of HIV-1 latency. Trends Mol Med 2004, 10:525-531]. These cells constitute another major reservoir of HIV-1.

In cell culture conditions, non-specific or bystander killing by the lentiviral vector have not been observed. In the absence of HIV-1, the Rev-dependent anlO vector can be stably maintained in the healthy cell population for extended periods of time (FIG. 4E), an indication of lack of anlO gene expression and cytotoxicity in the absence of Rev.

### EXAMPLES

Cloning of the anlO gene from B. anthracis: pNL-GFP-RRE-SA has been previously described. pNL-AnlO-GFP-RRE-SA was constructed by inserting the BamHI-Xhol fragment of pAnlO, a plasmid containing the anlO gene of the 34F2 (Sterne) strain of B. anthracis into the BamHI-Sall sites of pNL-GFP-RRE-SA. pNL-AnlO-RRE-SA was constructed by further deletion of the GFP ORF with restriction digestion. Successful cloning of the anlO gene was further confirmed by DNA sequence analysis. The packaging construct, pCMVΔ8.2, was obtained elsewhere. pCAGGSSF162gp160 was obtained from the NIH AIDS Research & Reference Reagent Program, NIAID, NIH.

Virus production: The HIV-1 strains, NL4-3.HSA.R+E−(VSV-G) and the replication-competent NL4-3.HSA.R+E+ (“R” represents the Vpr gene and “E” represents the viral envelope gene) were provided by the NIH AIDS Research & Reference Reagent Program, NIAID, NIH. In both viruses, the murine heat-stable antigen CD24 (HSA) gene was inserted into the nef region that allows HIV-1-positive cells to be monitored by surface staining of HSA. Viruses were produced by transfection of HEK293T cells (provided by the NIH AIDS Research & Reference Reagent Program, NIAID, NIH), using Lipofectamine™2 000. HIV-1 titer was determined using an indicator cell line, Rev-CEM, as previously described. The Rev-dependent GFP and AnlO lentiviruses, vNL-GFP-RRE-SA and vNL-AnlO-RRE-SA, were produced by cotransfection of HEK293T cells with calcium phosphate. Briefly, two million cells were cultured in a petri dish and cotransfected with 10 μg of either pNL-GFP-RRE-SA or pNL-AnlO-RRE-SA, 7.5 μg of pCMVΔ8.2, and 2.5 μg of the envelope constructs. Transfected cells were cultured overnight, and then the supernatant was removed and replaced with 10 ml fresh DMEM plus 10% heat-inactivated fetal bovine serum (FBS). For the production of vNL-AnlO-GFP-RRE-SA, 10 nM 6-boc orthinine amide-β-cyclodextrin was also added into the medium to prevent cell lysis by anlO expression. Viruses were harvested at 48 hours and then concentrated by multiple rounds of concentration through anion exchange columns and size exclusion columns. Concentrated virus was divided into 50 μl aliquots and stored at −80° C. Viral p24 level was determined using p24 ELISA assay (Beckman Coulter, Miami, Fla.). The p24 levels of concentrated viruses were between 2 and 10 μg/ml. The titer of vNL-GFP-RRE-SA was measured directly on an HIV-1-positive cell line, J1.1 (provided by the NIH AIDS Research & Reference Reagent Program, NIAID, NIH), which was cultured in 50 ng/ml PMA (phorbol myristate acetate) to stimulate HIV-1 activity. GFP-positive J1.1 cells were enumerated on FACSCalibur (BD Biosciences, San Jose, Calif.). The titer of vNL-AnlO-GFP-RRE-SA cannot be measured directly due to its cytolytic activity, and thus was estimated based on p24 levels, using the titer of vNL-GFP-RRE-SA as a reference.

Cells and viral infection: CEM-SS was acquired from the NIH AIDS Research & Reference Reagent Program, NIAID, NIH. Macrophages were differentiated from human monocytes from the peripheral blood of HIV-1 negative donors. Briefly, two million peripheral blood mononuclear cells were plated into each well of six plates in serum-free RPMI medium for one hour. Adherent cells were cultured in RPMI plus 10% FBS and 10 ng/ml macrophage colony stimulating factor (M-CSF) (R&D System, Minneapolis, Minn.) for two weeks with medium change every two days. Differentiated macrophages were infected with NL4-3.HSA.R+E−(VSV-G) at a multiplicity of infection of 0.1. Viral replication was monitored by cell surface staining of mouse CD24 antigen and p24 ELISA (Beckman Coulter, Miami, Fla.). CEM-SS T cells were infected with replication competent HIV-1 NL4-3.HSA.R+E+. Aliquots of infected cells were superinfected at 24 hours with vNL-I-RRE-SA using different doses of concentrated virus. HIV-1-positive cell were monitored by immunostaining and flow cytometry on a FACSCalibur (BD Biosciences, San Jose, Calif.).

Immunofluorescent staining: One half to one million infected cells were removed from the culture dish and washed once with cold PBS, centrifuged for 5 minutes at 400×g and resuspended in 400 μl cold staining buffer (PBS plus 1% BSA). Nonspecific binding was blocked by adding 5 μl Rat IgG (10 mglml). HIV-1-positive cells were stained with 2 μl of PE-labeled Rat Anti-Mouse CD24. For isotype control staining, PE-labeled Rat IgG₂ₐ was used. Stained cells were incubated on ice for 30 minutes and then washed with cold PBS plus 1% BSA and resuspended in 500 μl of 1% paraformaldehyde for flow cytometry analysis on a FACSCalibur (BD Biosciences, San Jose, Calif.).

PCR amplification: Total cellular DNA was purified using a Wizard Genomic DNA purification kit (Promega, Madison, Wis.). For the detection of the AnlO lentiviral vector in infected cells by PCR, the forward primer 5′GGTTAGACCAGATCTGAGCCTG3′ (SEQ ID NO:1) and the reverse primer 5′GTGTTTCTGCCATGGTAAGG 3′ (SEQ ID NO:2) were used. PCR was carried out in 1× Ambion PCR buffer, 125 μM dNTP, 50 pmol each primer, 1 U SuperTaq Plus (Ambion Inc. Austin, Tex.) with 35 cycles at 94° C. for 10 seconds, 68° C. for 50 seconds. For relative quantification of the PCR reaction, the cellular β-actin pseudogene was also amplified with primers from the QuantumRNA β-actin Internal Standards (Ambion Inc. Austin, Tex.). Briefly, the PCR was carried out in 1× Ambion PCR buffer, 125 μM dNTP, 1 U SuperTaq Plus with 25 cycles at 94° C. for 20 seconds, 68° C. for 60 seconds.

Modes of treatment of patients infected with HIV will include injecting viral particles into patients. The dosage will, of course, depend on the extent of infection.

1. An isolated nucleic acid molecule comprising:
a) a promoter, wherein the activity of the promoter is dependent on the presence of the human immunodeficiency virus (HIV) Tat protein;
b) at least one splice donor site and at least one splice acceptor site;
c) an expressible sequence which is not a wild-type HIV sequence, wherein at least part of the expressible sequence is located in an intron between the splice acceptor site and the splice donor site;
and
d) a Rev Responsive Element (RRE) from the human immunodeficiency virus,
wherein: (i) elements (a)-(d) are operably linked,
(ii) the expressible sequence comprises a therapeutic gene, or a complement thereof, and
(iii) the therapeutic gene or complement thereof encodes a cytotoxic, cytolytic or cell apoptosis inducing protein; or a protein stimulate immune response to HIV infection; or a complement thereof.

2. The nucleic acid molecule of claim 1 wherein said cytotoxic, cytolytic or cell apoptosis inducing protein is anthrolysin O.

3. A vector containing the nucleic acid molecule of claim 1.

4. The vector of claim 3, additionally comprising a recombinant virus.

5. The vector of claim 3, wherein said virus is replication incompetent.

6. The vector of claim 4 wherein said virus is a recombinant retrovirus.

7. The vector of claim 6 wherein said retrovirus is a recombinant lentivirus.

8. A host cell containing a nucleic acid molecule of claim 1.

9. A method of determining whether HIV is present in a sample comprising:
a) contacting the host cell of claim 8 with the sample;
b) culturing the cell for an amount of time sufficient to allow HIV infection and gene expression; and
c) determining whether the reporter gene is expressed by the cell;
wherein expression of the expressible sequence is indicative of the presence of HIV in the sample.

10. The method of claim 9, conducted in the presence of β-cyclodextrin or a derivative thereof.

11. A method of determining whether a cell is infected with HIV comprising:
a) contacting the cell with the vector of claim 3;
b) culturing the cell for an amount of time sufficient to allow HIV gene expression;
and
c) determining whether the expressible sequence is expressed by the cell;
wherein expression of the expressible sequence is indicative of HIV infection of the cell.

13. A method of determining whether a subject is infected with HIV comprising:
a) contacting the cells of the subject with the virus vector of claim 3;
and
b) determining whether the expressible sequence is expressed by the cells;
wherein expression of the expressible sequence is indicative of HIV infection.

14. A method of killing a cell infected with HIV comprising contacting the cell with the virus vector of claim 3.

15. A method of treating a subject infected with HIV comprising administering to the subject the virus vector of claim 3.

16. A method of determining whether a compound is capable of killing an HIV-infected cell comprising contacting the HIV-infected cell with the vector of claim 3 wherein the therapeutic gene or complement thereof encodes the test compound and determining whether the encoded test compound kills the HIV-infected cell.

17. A pharmaceutical composition comprising an effective amount of the vector of claim 3 and a pharmaceutically acceptable carrier therefore.


SEQUENCE LISTING (2 sequences)

SEQ ID NO: 1 - DNA, 22 bp, Artificial Sequence
  Forward primer for amplification of AnlO vector sequence
ggttagaccagatctgagcctg

SEQ ID NO: 2 - DNA, 20 bp, Artificial Sequence
  Reverse primer for amplification of AnlO vector sequence
gtgtttctgccatggtaagg